(19) United States
(12) Patent Application Publication
(10) Pub. No.: US 2007/0212309 A1
(43) Pub. Date: Sep. 13, 2007

(54) HAIR MOISTURIZING PROCESS

(76) Inventor: Kathryn LaNae Williams Pruess, Salt Lake City, UT (US)

Correspondence Address: ADVANTIA LAW GROUP, 9035 SOUTH 1300 EAST, SUITE 200, SANDY, UT, 84094, US

(21) Appl. No.: 11/308,145

(22) Filed: Mar. 8, 2006

(51) Int. Cl. A61K 8/18

(52) U.S. Cl. 424/47; 132/200

(57) ABSTRACT

A method of moisturizing hair comprising the steps of cooling a hair care product and applying the cooled hair care product to the hair. The hair care product may be cooled before it is applied to the hair and/or as it is applied to the hair. The hair care product may be cooled to below a temperature of the surrounding air. The hair may be cooled to below a temperature of the surrounding air.

### BACKGROUND OF THE INVENTION

1. Field of the Invention

The present invention relates generally to hair moisturizing processes and, more particularly, to a method of moisturizing the hair and/or sealing moisture into the hair by applying a cooled moisturizing hair product to the hair.

2. Description of the Related Art

Heating hair has been found to have a damaging effect on hair, particularly when a moisturizing product has not been applied to the hair. Cooling the hair without a moisturizing product has also been found to damage the hair. It has been suggested that to lock in moisture, a cool rinse after applying a moisturizing product to the hair. However, many people use a moisturizing product such as a conditioner while showering. To some, a cool rinse of the hair at the end of the shower is undesireable. Even if a cool rinse is used, there may be more that can be done to moisturize the hair and/or lock moisture into the hair from products used after the shower, or without a shower during styling. Further, if extra conditioning is not needed, and a cool rinse is not tolerated by a person, then the current state of the art does not adequately address the problem of moisturizing and/or locking in moisture to the hair.

The current practice of drying hair is to apply a heated stream of air to the hair until moisture is dissipated from the hair. Moisture, however, may be needed for healthy hair. It is believed, but not meant to be limited, that hair fiber may be constructed of molecular chains of atoms which are folded upon themselves much like accordion pleats. Some hydrogen bonding may occur between the molecules, and between different portions of the same molecules, and that this bonding is responsible for the characteristic known as hair setting. Water may soften hair to such an extent that it can be stretched when totally wet up to several times its initial length without breaking. Furthermore, there is no critical point of full wetness which must be attained before the hair can be so influenced. The ability of hair to be stretched may increase in proportion to the amount of water it has absorbed, either from liquid water, or from water vapor present in the air.

The general structure of a human hair is a cortex surrounded by a cuticle. The cortex is where the moisture of the hair may be found. The cortex includes strands of keratin, lying along the length of the hair. These keratin fibers include low-sulfur keratins which are compressed into bundles of larger fibers, and are held together by a mass of sulfur-rich keratins. The cortex may also include pigment (melanin).

The cuticle includes several (from about 6 to about 10) overlapping layers of long cells. Each cell is about 0.3 μm thick and around 100 μm long. These cells lie along the axis of the cortex as tiles lie on the roof of a house, such that the exposed edge is closer to the end of the hair, and the covered edge is closer to the follicle.

As the hair ages, or is damaged, the cuticle layers are damaged and fall off of the hair. At times, the hair becomes so damaged that all of the cuticle layers are removed from the cortex, and the cortex is exposed. When hair is dried by heat, the cuticle layers tend to stand up, or position themselves more perpendicular to the cortex. When the cuticle is in this position, the hair is less manageable, more susceptible to damage, is less effective at retaining moisture, is less smooth, and exhibits dryness, dullness, brittleness, and/or “frizzyness”.

Some hair care products help the layers of the cuticle to lay flat, thus reducing moisture loss of the hair, making the hair more manageable, smooth, and reducing “frizzyness.” Conditioners help the layers of the cuticle lay flat by providing large molecules with a positive charge (hairs carry a small negative charge), that cling to the hair, smoothing over the cuticle layers, filling in breaks, and so forth. Proteins, dimethicone, and panthenol are also helpful in hair care products. Panthenol, for example, may be absorbed into the shaft and provide moisture to the cortex.

Wetting or hydration of the hydrogen bonds referred to above releases the normal hydrogen bonding attachments within the hair fiber. As a result, moisturization will relax the hair molecular structure by causing the hydrogen bonds to release their hold, allowing the pleats to unfold and the molecules to change position and to bypass each other. One might visualize the hair as a resilient system of fibrous molecules packed together much like the fibers of a strand of hemp rope. Under ambient conditions this system comprises a natural inter-molecular hydrogen bonding mixture of attached hydrogen bonds in equilibrium with some that are not attached. The ratio of the attached to unattached bonds also depends in part on the number of available water molecules within the system. The greater the number of attached bonds (as in dry hair) the harder and stiffer is the system. As more of these hydrogen bonds become unattached (as in moisturized, wetter hair) the system becomes more flexible and more easily stretched.

Several attempts have been made to provide hair care products, processes, and apparatuses that introduce moisture into the hair. For example, U.S. Pat. No. 3,974,840 to Doyle discloses a portable combination moisturizer and dryer which includes a base, a head supporting assembly, and a head assembly into which the rest of the unit can nest for carrying and for storage. The head assembly includes a generally annular air distribution plenum for receiving moisture-laden or drying air and directing it radially inwardly. In a preferred embodiment, a squirrel-cage blower is contained in an impeller housing disposed in the lower portion of the head supporting assembly, which lower portion is mounted for adjustable pivotal movement with respect to the base. The upper portion of the head supporting assembly terminates in a horizontally disposed manifold constituting a portion of the plenum, to provide for passage of either moisture-laden air or drying air through the head mounting assembly and into the interior of the plenum, while also permitting adjustment of the position of the head assembly with respect to the head mounting assembly. The intermediate portion of the head mounting assembly is divided into two separate sections. The air heater is provided in one of the sections which defines the impeller-driven air conduit, and a steam generator is provided in the other section, along with means for directing the generated steam into the horizontal manifold.

Furthermore, Vaiano discloses, in U.S. Pat. No. 3,863,651 an apparatus for washing, massaging and conditioning hair. The housing, a rigid outer shell, comprises an inner baffle shell provided with means for creating a turbulence when water under pressure is passed therethrough. The inner shell is provided with an elastic band adapted to seal the inner shell watertight when stretched about a user's head. Means for supplying the shells with water under pressure are provided. The apparatus further comprises means for drying and means for conditioning the hair. Automatic switching means for operating the washing, drying and hair conditioning means, in timed relationship are provided.

The existing art, however, does not address the problem of moisturizing the hair and sealing the moisture into the hair. The prior art further does not adequately address the problem of locking in moisturizing products after/outside of the shower.

What is needed is a hair moisturizing technique that solves one or more of the problems described herein and/or one or more problems that may come to the attention of one skilled in the art upon becoming familiar with this specification.

### SUMMARY OF THE INVENTION

The present invention has been developed in response to the present state of the art, and in particular, in response to the problems and needs in the art that have not yet been fully solved by currently available hair moisturizing techniques. According, to one embodiment, the present invention has been developed to provide a method of moisturizing hair comprising the steps of: cooling a hair care product; and applying the cooled hair care product to hair. The hair care product may be cooled as it is applied to the hair. The hair care product may be cooled before it is applied to the hair.

According to one embodiment, the cooling the hair care product may be by rapid expansion. The cooling hair care product may be by heat exchange with a fluid.

In another embodiment, the hair care product may be cooled to below the temperature of air surrounding the hair.

According to one embodiment, the hair care product may be one selected from the group consisting of: shampoo, conditioner, spray, mousse, gel, straightening gel, forming gel, glaze, hair oil, hair dye, texture modifier, foam, hair cream, pomade, moisturizer, paste, anti-frizz, leave-in treatment and combinations thereof. The hair care product may be a leave-in treatment.

In yet another embodiment, the method may further include the step of cooling the hair. The cooling the hair may be cooling the hair to below a temperature of air surrounding the hair.

According to a further embodiment, the present invention provides a method of decreasing damage to hair from drying including the step of sealing moisture in the hair. The step of sealing moisture in the hair may include applying a cooled hair care product to the hair. The hair care product may be cooled to a temperature of less than a temperature of air surrounding the hair. The hair care product may be cooled as it is applied to the hair. The hair care product may be cooled before it is applied to the hair.

The cooling the hair care product may be by rapid expansion. The cooling hair care product may be by heat exchange with a fluid. The hair care product may be cooled in a refrigerator.

The hair care product may include one selected from the group consisting of: shampoo, conditioner, spray, mousse, gel, straightening gel, forming gel, glaze, hair oil, hair dye, texture modifier, foam, hair cream, pomade, moisturizer, paste, anti-frizz, leave-in treatment and combinations thereof. The hair care product may be a leave-in treatment.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussion of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize that the invention can be practiced without one or more of the specific features or advantages of a particular embodiment. In other instances, additional features and advantages may be recognized in certain embodiments that may not be present in all embodiments of the invention.

These features and advantages of the present invention will become more fully apparent from the following description and appended claims, or may be learned by the practice of the invention as set forth hereinafter.

### DETAILED DESCRIPTION OF THE INVENTION

For the purposes of promoting an understanding of the principles of the invention, reference will now be made to the exemplary embodiments illustrated in the drawings, and specific language will be used to describe the same. It will nevertheless be understood that no limitation of the scope of the invention is thereby intended. Any alterations and further modifications of the inventive features illustrated herein, and any additional applications of the principles of the invention as illustrated herein, which would occur to one skilled in the relevant art and having possession of this disclosure, are to be considered within the scope of the invention.

Reference throughout this specification to “one embodiment,” “an embodiment,” or similar language means that a particular feature, structure, or characteristic described in connection with the embodiment is included in at least one embodiment of the present invention. Thus, appearances of the phrases “one embodiment,” “an embodiment,” and similar language throughout this specification may, but do not necessarily, all refer to the same embodiment, different embodiments, or component parts of the same or different illustrated invention. Additionally, reference to the wording “an embodiment,” or the like, for two or more features, elements, etc. does not mean that the features are related, dissimilar, the same, etc. The use of the term “an embodiment,” or similar wording, is merely a convenient phrase to indicate optional features, which may or may not be part of the invention as claimed.

Each statement of an embodiment is to be considered independent of any other statement of an embodiment despite any use of similar or identical language characterizing each embodiment. Therefore, where one embodiment is identified as “another embodiment,” the identified embodiment is independent of any other embodiments characterized by the language “another embodiment.” The independent embodiments are considered to be able to be combined in whole or in part one with another as the claims and/or art may direct, either directly or indirectly, implicitly or explicitly.

As used herein, “comprising,” “including,” “containing,” “is,” “are,” “characterized by,” and grammatical equivalents thereof are inclusive or open-ended terms that do not exclude additional, unrecited elements or method steps. “Comprising” is to be interpreted as including the more restrictive terms “consisting of” and “consisting essentially of.”

As used herein, “dew point” means the temperature to which a parcel of air must be cooled at a constant barometric pressure for water vapor to condense out of the air.

As used herein, “fluid” means a phase of a substance wherein the substance has the ability to flow. Fluids include the phases of liquid, gas, and plasma.

Finally, the fact that the wording “an embodiment,” or the like, does not appear at the beginning of every sentence in the specification, such as is the practice of some practitioners, is merely a convenience for the reader's clarity. However, it is the intention of this application to incorporate by reference the phrasing “an embodiment,” and the like, at the beginning of every sentence herein where logically possible and appropriate.

One object of the present invention is to provide a process for moisturizing hair and/or sealing the moisture into the hair by assisting the cuticle layers to lay more flat. The method of this invention may be used in conjunction with other hair care products, techniques, and methods for moisturizing, finishing, managing, conditioning, and treating hair. The method of the present invention may be used to enhance the beneficial effects of hair care products, particularly those that moisturize hair, assist in repairing hair, modify the look of hair, and the like.

It has been found that cooling hair by applying a moisturizing product has been cooled has beneficial effects for the hair. For example, cooling the hair may help the cuticles to lay flat and/or close. Further, cooling the hair may seal the moisturizing product into the hair. As a result, cooling the hair has beneficial effects to the hair. Further, it has been observed that hair that is treated with heat after a moisturizing product has been applied to the hair may reduce the beneficial effects of the moisturizing product. However, if a cooled hair care product is applied to the hair, the beneficial effects of the moisturizing product are not reduced, and may be enhanced.

The present invention includes a method of moisturizing hair. The method includes the step of cooling the hair. The cooling the hair may take place by applying a cooled hair care product to the hair. As has been described, cooling the hair by applying a cooled hair care product to the hair may have the effect of sealing or locking the moisture into the hair. Further, the cooling the hair by applying a cooled hair care product to the hair may have the effect of reducing “frizzyness”, and/or smoothing the hair by assisting the cuticle layers to lay closer to the cortex of the hair.

The present invention provides a method for moisturizing hair or locking moisture into the hair. In one embodiment, the hair may be moisturized by condensing water on the hair from the surrounding air. It is theorized, but not meant to be limiting to the present invention, that a surface with a temperature below that of the temperature of the surrounding air will exchange heat with the air immediately surrounding the surface. If the air surrounding the surface has water vapor, and the dew point of the water vapor in the air is above room temperature, the water vapor is not likely to condense out of the air. However, if the air immediately surrounding the surface is cooled to below the dew point of the water vapor in the air, the water vapor will likely condense out of the air onto the surface. Thus, if hair is cooled to below the dew point of the air surrounding the hair, and it in turn cools the air surrounding it to below the dew point, then water vapor will likely condense onto the hair, moisturizing the hair. According to the present invention, the temperature of the hair may be cooled to below the temperature, and/or below the dew point of the surrounding air by applying a hair care product to the hair, wherein the hair care product has been cooled to below the temperature and/or below the dew point of the surrounding air.

It is further theorized that such condensation evenly distributes water condensation onto the hair that is cooled to below the dew point of the surrounding air without manually distributing the moisture to the hair. It should be noted that when hair care products are applied to the hair, they may not evenly distributed to the hair. For example, hair care products that are sprayed onto the hair are applied in droplets only to the surface of the hair directed toward the sprayer. Thus, the present invention includes a method of evenly moisturizing the hair by cooling the hair to below room temperature. In another embodiment, the hair is cooled to at or below the dew point of the air surrounding the hair. The hair may be cooled by applying a cooled hair care product to the hair. The hair care product may be cooled to below room temperature. The hair care product may be cooled to below the dew point of the air surrounding the hair. Thus the hair may be cooled to below room temperature, or below the dew point of the air surrounding the hair.

The hair care products of the present invention may include, for example, shampoo, conditioner, spray, mousse, conditioning mousse, gel, straightening gel, forming gel, glaze, hair oil, hair dye, texture modifier, foam, hair cream, pomade, moisturizer, paste, anti-frizz, leave-in treatment, and combinations thereof. In one embodiment, the hair care product is a moisturizer. In one particular embodiment, the hair care product is a moisturizer to be left in the hair such as the one sold under the tradename Infusium 23® by Procter and Gamble (Cincinnatti, Ohio). In another particular embodiment, the hair care product is a conditioning mousse such as one sold as 3-in-1 Mousse, Maximum Hold, by Clairol (a division of Procter & Gamble, Stamford, Conn.).

In one embodiment, the hair care product is cooled before it is applied to the hair. The hair care product may be cooled by any process known in the art. The hair care product may be cooled by heat exchange with a cooled fluid. The hair care product may be cooled by heat exchange with a Peltier junction. The hair care product may be cooled by rapid expansion.

In another embodiment, the hair care product is cooled as it is applied to the hair. The hair care product may be cooled by any process known in the art. The hair care product may be cooled by heat exchange with a cooled fluid. The hair care product may be cooled by heat exchange with a Pielter junction. The hair care product may be cooled by rapid expansion.

The method of cooling the hair care product by heat exchange with another fluid is the basic method used by an air conditioner to cool air. In this process, a fluid, such as R-34, Freon, and so forth, is compressed using a compressor. The fluid may be compressed from the gas phase to the liquid phase. The compressed fluid increases in temperature, and so may pass through a heat exchanger wherein the surrounding air is heated by the compressed fluid, and the compressed fluid is in turn cooled by the surrounding gas. The cooled and compressed fluid is then allowed to expand. The expansion may expand the fluid from the liquid phase to the gas phase, or from a higher-pressure gas phase to a lower-pressure gas phase. As the fluid expands, it looses energy, thus becoming cooler. The expanded, cooled fluid passes through a heat exchanger wherein the hair care product is cooled by the expanded fluid, and the expanded fluid is heated by the hair care product. The hair care product may loose enough energy to be colder than room temperature, or colder than the dew point of the surrounding air, or well below the dew point of the surrounding air. The heated and expanded fluid may then be cycled again into the compressor, starting the cycle again.

The cooled hair care product may be cooled to a temperature of below the temperature of the surrounding air. The cooled hair care product may have a temperature of below the dew point of the surrounding air. Particularly as the dew point of the surrounding air approaches the temperature of the surrounding air, the temperature of the cooled hair care product may be below, or well below the dew point of the surrounding air.

The hair care product may be cooled by thermoelectric cooling. One thermoelectric cooling process is known as the Peltier effect. The Peltier effect occurs when a current is passed through two dissimilar metals or semiconductors (n-type and p-type). The dissimilar metals or semiconductors may be connected together at two junctions, known as Peltier junctions. As the current passes through the junctions, it causes one junction to loose heat (cool) and the other to gain heat. The cooled junction may then be used in a heat exchanger whereby the hair care product is cooled by the cooled junction, and the cooled junction is in turn heated by the hair care product. Further, a fluid may be cooled by heat exchange with the cooled junction, and the cooled fluid may then be heated by cooling the hair care product through a heat exchanger. The cooled hair care product may have a temperature of below the temperature of the surrounding air. The cooled hair care product may have a temperature of below the dew point of the surrounding air. Particularly as the dew point of the surrounding air approaches the temperature of the surrounding air, the temperature of the cooled hair care product may be below, or well below the dew point of the surrounding air.

In another example, the hair care product may be cooled by rapid gas expansion. As a compressed gas rapidly expands to a lower pressure, the temperature of the gas decreases. In one embodiment, the pressure of the compressed gas is well above that of the surrounding atmosphere. The gas is then allowed to expand rapidly to the pressure of the surrounding atmosphere. The hair care product may be cooled by heat exchange with the rapidly expanding gas.

In one embodiment, the hair care product may be propelled along with the rapidly expanding gas toward the hair. As it is propelled with the rapidly expanding gas, the hair care product is cooled by the rapidly expanding gas.

In another embodiment, the hair care product is cooled by a rapidly expanding gas flowing past a surface that contacts the hair care product. For example, the hair care product may be contained in a canister through which heat exchange is possible, such as a thin-walled metal canister. The rapidly expanding gas may be directed to pass over the canister, thus exchanging heat with, and cooling the hair care product. In one embodiment, the canister holds only a single use amount of hair care product. The canister may be any shape, but the larger the ration of surface exposed to the rapidly expanding gas to the volume of the canister, the less rapidly expanding gas is needed to cool the hair care product.

The greater the pressure difference between the expanded gas and the compressed gas, the greater the temperature difference will be between the expanded gas and the compressed gas will be. As a result, if a compressed gas is allowed to exchange heat with the surrounding air, it will eventually become substantially the same temperature as the surrounding air. As it expands, then, the expanded gas will have a temperature of below the temperature of the surrounding air. Further, the expanded gas may have a temperature of less than the dew point of the surrounding air. Particularly, as the dew point of the surrounding air approaches the temperature of the surrounding air, the temperature of the expanded gas may be well below the dew point of the surrounding air. Further still, as the expanded gas cools the hair toward which it may be directed, the hair may be cooled to lower than the temperature of the surrounding air, or below the dew point of the surrounding air. Thus, as the hair is cooled to below the temperature of the surrounding air, or below the dew point of the surrounding air, water may condense from the surrounding air onto the hair, moisturizing the hair.

In still another embodiment, the hair care product may be cooled by the phase change of a second substance. The second substance may make a phase change that absorbs energy, such as a change from a solid to a liquid, solid to gas, solid to plasma, liquid to gas, liquid to plasma, or gas to plasma. For example, when water changes phase from a liquid to a gas by evaporation, the gaseous phase has more energy than did the liquid phase, and so it must absorb energy. The energy may be absorbed from the surroundings. This is the principal behind how a swamp cooler cools air. In one embodiment, the second substance undergoes a phase change. In one embodiment, energy from air surrounding the second substance is absorbed by the substance, thus decreasing the temperature of the air. The cooled air then exchanges heat with, and thus cool the hair care product. The secondary substance may be water, changing phase from liquid to a gas.

In any of the above embodiments, a second fluid may be cooled by the cooling process. The cooled fluid may in turn heat exchange with the hair care product to cool the hair care product. For example, the cooling process may cool air in a closed system, such as a refrigerator. The hair care product may be placed in the closed system with the cooled air. The cooled air then exchanges heat with the hair care product, thus cooling the hair care product. The second fluid may be cooled by rapidly expanding the second fluid, heat exchange with a Peltier junction, heat exchange with an expanding fluid, or so forth.

Further, the cooling process may include cooling a reservoir. The reservoir may include a fluid. The reservoir may be cooled by any process herein described. The hair care product may in turn be cooled by heat exchange with the cooled reservoir. The reservoir may capable of absorbing sufficient energy from the hair care product such that the hair care product is cooled to below the room temperature, or below the dew point of the air surrounding the hair. Further, the reservoir may be continually cooled, even while the hair care product is exchanging heat with the reservoir. The reservoir may be cooled by continuously pumping the fluid of the reservoir through a heat exchanger to cool it, and then back into the reservoir. For example, the reservoir may be the air inside of a refrigerator. The hair care product may then be placed inside of the refrigerator to heat exchange with the air, thus cooling the hair care product.

In yet another embodiment, the hair care product may be cooled by a chemical reaction in the hair care product. The cooling in this embodiment may occur before the product is applied to the hair, during application, or after application to the hair. In one example, a chemical reaction in the hair care product is initiated when the hair care product is exposed to the air. For example, oxygen in the air may initiate or be a reactant in a reaction in the hair care product that causes a phase change that absorbs energy from the hair care product and/or the hair, thus resulting in a decrease in the temperature of the hair care product. In another example, a second chemical may be mixed with the hair care product, and react with the hair care product to decrease the temperature of the hair care product. Such endothermic reactions are known. Such reactions may result in a phase change, or may result in a chemical reaction. One such endothermic reaction is a saponification reaction wherein an ester is hydrolyzed under basic conditions to form an alcohol and the salt of an acid. In another known endothermic reaction, a supersaturated liquid is mixed with a seed crystal of the solute, resulting in a phase change of the solute from a dissolved state to a solid state.

In another embodiment, the present invention includes the method of touching-up hair that has become dull, dry, brittle, and/or frizzy. At times, throughout the day, hair may loose its moisture, and exhibit these characteristics. The hair may be touched-up by applying a cooled gas to the hair. Any of the herein-mentioned methods and/or apparatuses may be used to touch-up the hair.

It is understood that the above-described embodiments are only illustrative of the application of the principles of the present invention. The present invention may be embodied in other specific forms without departing from its spirit or essential characteristics. The described embodiment is to be considered in all respects only as illustrative and not restrictive. The scope of the invention is, therefore, indicated by the appended claim rather than by the foregoing description. All changes which come within the meaning and range of equivalency of the claims are to be embraced within their scope.

The hair care product may be cooled by a combination of the processes mentioned herein. For example, a container of a hair care product with a compressed gas propellant may be placed in a refrigerator and cooled. The hair care product may then be further cooled by allowing the hair care product to exchange heat with the rapidly expanding gas propellant as the gas propellant escapes the container, expanding and carrying the hair care product with it.

For example, although the above embodiments describe that a reservoir may be inside of a refrigerator, it is envisioned that any space capable of substantially containing the reservoir may be used. Further, it is envisioned that the phase of the second substance in the reservoir may be any capable of heat exchange with the hair care product. Thus, the reservoir may be contained in a device capable of containing the second substance

Thus, while the present invention has been fully described above with particularity and detail in connection with what is presently deemed to be the most practical and preferred embodiment of the invention, it will be apparent to those of ordinary skill in the art that numerous modifications, including, but not limited to, variations in size, materials, shape, form, function and manner of operation, assembly and use may be made, without departing from the principles and concepts of the invention as set forth in the claims.

What is claimed is:

1. A method of moisturizing hair, comprising the steps of:
cooling a hair care product; and
applying the cooled hair care product to hair.

2. The method of claim 1, wherein the hair care product is one selected from the group consisting of: shampoo, conditioner, spray, mousse, conditioning mousse, gel, straightening gel, forming gel, glaze, hair oil, hair dye, texture modifier, foam, hair cream, pomade, moisturizer, paste, anti-frizz, leave-in treatment and combinations thereof.

3. The method of claim 1, wherein the hair care product is a leave-in treatment.

4. The method of claim 1, wherein the hair care product is cooled as it is applied to the hair.

5. The method of claim 1, wherein the hair care product is cooled before it is applied to the hair.

6. The method of claim 1, wherein the hair care product is cooled after it is applied to the hair.

7. The method of claim 1, wherein the cooling hair care product is by heat exchange with a fluid.

8. The method of claim 1, wherein the hair care product is cooled to below the temperature of air surrounding the hair.

9. The method of claim 1, further comprising the step of cooling the hair.

10. The method of claim 9, wherein the cooling the hair includes cooling the hair to below a temperature of surrounding air.

11. A method of decreasing damage to hair from drying including the steps of:
moisturizing the hair; and
sealing the moisture into the hair.

12. The method of claim 11, wherein the step of sealing the moisture into the hair comprises applying a cooled hair care product to the hair.

13. The method of claim 12, wherein the hair care product comprises one selected from the group consisting of: shampoo, conditioner, spray, mousse, gel, straightening gel, forming gel, glaze, hair oil, hair dye, texture modifier, foam, hair cream, pomade, moisturizer, paste, anti-frizz, leave-in treatment and combinations thereof.

14. The method of claim 12, wherein the hair care product is a leave-in treatment.

15. The method of claim 12, wherein the hair care product is cooled to a temperature of less than a temperature of air surrounding the hair.

16. The method of claim 12, wherein the hair care product is cooled as it is applied to the hair.

17. The method of claim 12, wherein the hair care product is cooled before it is applied to the hair.

18. The method of claim 12, wherein the hair care product is cooled after it is applied to the hair.

19. The method of claim 12, wherein the cooling hair care product is by heat exchange with a fluid.

20. The method of claim 19, wherein the hair care product is cooled in a refrigerator.
